(19) United States
(12) Patent Application Publication
(10) Pub. No.: US 2024/0280902 A1
(43) Pub. Date: Aug. 22, 2024

(54) Compound Having Unsaturated Double Bond, Composition Comprising The Compound, Polybenzoxazole, And Polybenzoxazole Film For Semiconductor Device

(71) Applicant: Nippon Kayaku Kabushiki Kaisha, Tokyo (JP)

(72) Inventors: Naoki Kawamoto, Tokyo (JP); Firyon Ko, Tokyo (JP); Taihei Koumoto, Tokyo (JP)

(21) Appl. No.: 18/435,588

(22) Filed: Feb. 7, 2024

(30) Foreign Application Priority Data
Feb. 9, 2023 (JP) 2023-018629

(51) Int. Cl. G03F 7/031; C07C 215/80

(52) U.S. Cl. CPC G03F 7/031; C07C 215/80

(57) ABSTRACT

A compound having unsaturated double bonds of formula (1):

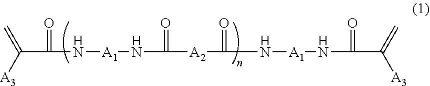

wherein A₁ represents a divalent linking group of formula (1-1), (1-2), or (1-3):

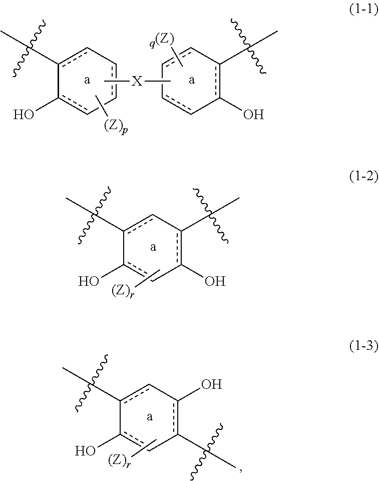

wherein ring a represents a benzene cyclohexane ring; X represents a direct bond or a divalent linking group; Z represents a monovalent substituent; p, q, and r are numbers of the monovalent substituent Z, p and q each independently represent an integer of 0 to 3, r represents an integer of 0 to 2,
or a divalent linking group other than formulae (1-1), (1-2), and (1-3), provided that at least one of A₁ is a divalent linking group of formula (1-1), (1-2), or (1-3); A₂ represents a divalent linking group; A₃ represents a hydrogen atom or a methyl group; and n is an average value of the repeating unit numbers, and is a real number within a range of 1≤n≤9).

### BACKGROUND OF THE INVENTION

### Technical Field

The present invention relates to a compound having unsaturated double bonds, a composition containing the compound, a polybenzoxazole, and a polybenzoxazole film for semiconductor device. A compound having unsaturated double bonds of the present invention can be applied to a surface protective film for a semiconductor device, an interlayer insulating film, an insulating film of a rewiring layer, or the like.

### Background Art

Conventionally, polyimide resins, polybenzoxazole resins, and the like, which are excellent in heat resistance, mechanical properties, etc., have been widely used for surface protective films, interlayer insulating films, etc. of semiconductor devices (see Patent Literature 1, JPH11-199557A). As an attempt to use a polyimide resin or a polybenzoxazole resin as a surface protective film or an interlayer insulating film, a method of forming a through hole or the like by means of etching with the use of a positive photoresist comprising such a resin has been known. However, this method has a problem that complicated steps such as application and peeling of a photoresist are required. Therefore, for the purpose of streamlining work processes, a heat-resistant material to which photosensitivity is imparted has been studied (see Patent Literature 2, JPH11-24271A).

A thin film of a polyimide resin or a polybenzoxazole resin that is excellent in heat resistance and mechanical properties can be generally obtained by thermal dehydration ring closure of a coating film of a precursor thereof, which requires a firing at a high temperature of about 350° C. However, for example, next-generation memories such as a MRAM (Magnetoresistive Random Access Memory; magnetoresistive memory) or resins used for sealing such memories tend to get degraded at high temperatures. Therefore, a polyimide resin or a polybenzoxazole resin used for a surface protective film of such an element, or a polyimide resin or a polybenzoxazole resin used for an interlayer insulating film of a fan-out wafer level package forming a rewiring structure on a sealing resin of the element is required to be capable of being cured by firing at a low temperature of about 225° C. or less so as to obtain various properties comparable to those of a conventional material fired at a high temperature of about 350° C.

In addition, a polyimide resin that has been conventionally employed needs to use a large amount of an organic solvent such as N-methyl-2-pyrrolidone in a development step thereof, which requires a high cost. In view of not only the necessity of cost reduction but also safety and the recent increase in environmental problems, there has been a demand for elimination of organic solvents. In response to these demands, there have been proposed methods of using various heat-resistant resin materials capable of developing images (being patterned) with a dilute aqueous alkali solution, similarly as the case of a photoresist; for example, a method of mixing polyamide acid with a compound having an amino group, an amide group, a urethane group, or the like, and heating the mixture after an exposure in the presence of a photoinitiator (see Patent Literature 3, JPH6-289626A), a method of mixing a quinone diazide with a salt of a polyamide acid and an amine compound having a phenolic hydroxyl group (see Patent Literature 4, JPH6-161102A), and a method of mixing a polyamide acid with a base generator such as nifedipine (see Patent Literature 5. JPH5-5995A).

Each of these methods uses a positive photosensitive composition based on polyamide acid. Such a photosensitive composition exhibits relatively good developability, but has a small difference in solubility between an exposed portion and an unexposed portion, resulting in large film loss in patterns, and insufficient photosensitivity. In addition, these compositions have a disadvantage that because a large amount of free carboxylic acid is present in the polymer backbone, the backbone is unfavorably hydrolyzed over time by acidity of the polymer itself, and storage stability is extremely low.

Patent Literature 6, JPH2-37934B suggests a negative photosensitive material which is obtained by blocking a hydroxyl group with an intermolecular cyclic anhydride, the hydroxyl group being derived from an epoxy ring generated when glycidyl methacrylate interacts with a carboxy group of a polyamide acid to introduce a photosensitive group via an ester bond. However, the photosensitive material of the patent literature has a concern of low storage stability which is caused by influence of over time hydrolysis of the backbone and the photosensitive side chain, due to a large amount of free carboxylic acid in the polymer. In addition, this photosensitive material has had such a problem that an imidization reaction disadvantageously proceeds due to the heating at the time of introducing a photosensitive group, by which it has been hard to obtain a target polymer.

Moreover, with regard to communication terminals represented by smartphones, quantity of data communication has been continuously increasing, which requires higher communication frequency in order to transmit the data quantity in a short time. To increase the communication frequency, it is necessary to suppress transmission loss, which requires a material having a low dielectric constant and a low dielectric dissipation factor. However, it has been difficult to achieve both the properties as described above and the dielectric properties at a time.

In addition, according to miniaturization of interlayer insulating films, resolution of photosensitive material is getting more important. Having high resolution can lead to miniaturization and high functionality of electronic members.

### CITATION LIST

### Patent Literatures

PATENT LITERATURE 1: JPH11-199557A
PATENT LITERATURE 2: JPH11-24271A
PATENT LITERATURE 3: JPH6-289626A
PATENT LITERATURE 4: JPH6-161102A
PATENT LITERATURE 5: JPH5-5995A
PATENT LITERATURE 6: JPH2-37934B

### SUMMARY OF THE INVENTION

### Technical Problem

An object of the present invention is to provide a novel compound having unsaturated double bonds.

A further object of the present invention is to provide a composition containing the novel compound, a polybenzoxazole, and a semiconductor device.

Another object of the present invention is to provide a compound which is excellent in developability and resolution when a dilute aqueous alkali solution is used, and being capable of forming a cured film (preferably, a cured film excellent in thermal properties and electrical properties) even by heat treatment at a low temperature of 225° C. or lower (or, even by light irradiation and heat treatment at 225° C. or lower).

### Solution to Problem

As a result of extensive studies, the present inventors have found that it is possible to obtain a cured film containing a polybenzoxazole which is an intramolecular dehydrated ring-closed product thereof, even by a heat treatment at a low temperature of 225° C. or lower (or, even by a light irradiation and a heat treatment at 225° C. or lower), when a polymer (heat-resistant resin) of a compound with a specific structure having unsaturated double bonds is used. The present inventors have further found that it is possible to obtain a composition excellent in resolution while retaining the above-mentioned properties, when the compound has, in the molecule, a constituent unit(s) composed of a combination of a diamine-derived moiety and a dicarboxylic acid-derived moiety with a repetition number (n) controlled within a specific range.

That is, aspects and preferred embodiments of the present invention can be summarized as follows.

[1].

A compound having unsaturated double bonds represented by the following formula (1):

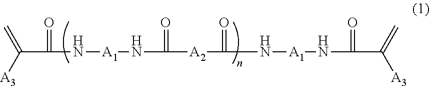

wherein A₁ represents a divalent linking group represented by the following formula (1-1), (1-2), or (1-3):

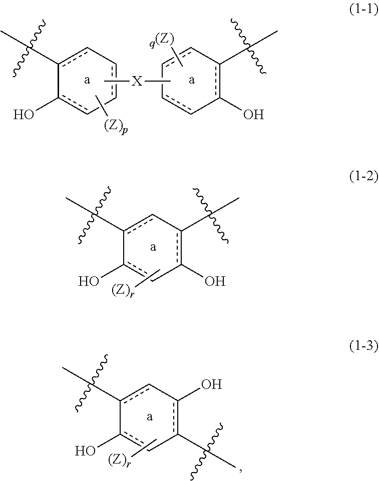

wherein ring a represents a benzene ring or a cyclohexane ring; X represents a direct bond or a divalent linking group, and when two or more Xs are present in the formula (1), the two or more Xs may be the same or different from each other; Z represents a monovalent substituent bonded to ring a, and when two or more Zs are present in the formula (1), the two or more Zs may be the same or different from each other; p, q, and r are numbers of the monovalent substituent Z, p and q each independently represent an integer of 0 to 3, r represents an integer of 0 to 2, and when two or more ps are present in the formula (1-1), the two or more ps may be the same or different from each other, and when two or more qs are present in the formula (1-1), the two or more qs may be the same or different from each other, and when two or more rs are present in the formulae (1-2) and (1-3), the two or more rs may be the same or different from each other,
or a divalent linking group other than the formulae (1-1), (1-2), and (1-3), provided that at least one of A₁ is a divalent linking group represented by the formula (1-1), (1-2), or (1-3), A₂ represents a divalent linking group obtained by removing two carboxy groups from a saturated aliphatic dicarboxylic acid compound, a divalent linking group obtained by removing two carboxy groups from an aromatic dicarboxylic acid compound, or a divalent linking group other than a divalent linking group obtained by removing two carboxy groups from a saturated aliphatic dicarboxylic acid compound or a divalent linking group obtained by removing two carboxy groups from an aromatic dicarboxylic acid compound, and when two or more A₂s are present, the two or more A₂s may be the same or different from each other; A₃ represents a hydrogen atom or a methyl group; and n is an average value of the repeating unit numbers, and is a real number within a range of 1≤n≤9).
   [2].

The compound having unsaturated double bonds according to [1], wherein A₁s are each independently a divalent linking group represented by any one of formulae (1-1), (1-2), and (1-3).

[3].

The compound having unsaturated double bonds according to [1] or [2], wherein all of ring(s) a is a benzene ring in the formulae (1-1), (1-2), and (1-3).

[4].

The compound having unsaturated double bonds according to any one of [1] to [3], wherein 1.0<n≤4.0.

[5].

The compound having unsaturated double bonds according to any one of [1] to [4], wherein at least one of A₂(s) is a divalent linking group obtained by removing two carboxy groups from a saturated aliphatic dicarboxylic acid compound.

[6].

The compound having unsaturated double bonds according to any one of [1] to [5], wherein each of A₂(s) is a divalent linking group obtained by removing two carboxy groups from a saturated aliphatic dicarboxylic acid compound.

[7].

The compound having unsaturated double bonds according to any one of [1] to [6], wherein each of A₂(s) is a divalent linking group obtained by removing two carboxy groups from a saturated aliphatic dicarboxylic acid compound, and the average number of carbon atoms contained in the aliphatic chain of each A₂ is 2 to 10.

[8].

The compound having unsaturated double bonds according to any one of [1] to [7], wherein X is each independently a direct bond or a divalent linking group represented by any one of the following formulae (a) to (f).

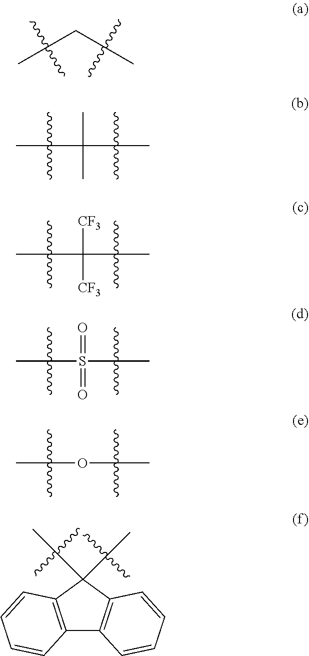

[9].

A composition containing the compound according to any one of [1] to [8] and a photopolymerization initiator or a curing catalyst.

[10].

A composition containing the compound having unsaturated double bonds according to any one of [1] to [8] and a compound capable of reacting with an unsaturated double bond.

[11].

A polybenzoxazole obtainable by heating a compound according to any one of [1] to [8], which is an intramolecular dehydrated ring-closed product of a polymer of the compound.

[12].

A semiconductor device provided with a surface protective film, an interlayer insulating film, or an insulating film of a rewiring layer, which contains the polybenzoxazole according to [11].

[13].

A dry film resist, which is obtainable by sandwiching the composition according to [9] or [10] between substrates.

### Advantageous Effects of Invention

The compound having unsaturated double bonds of the present invention can be developed with an alkaline aqueous solution and has excellent resolution. By using a polymer formed from the compound, a cured film containing polybenzoxazole can be obtained even by a heat treatment at a low temperature of 225° C. or lower (or, even by a light irradiation and a heat treatment at 225° C. or lower). The cured film according to a preferred embodiment of the present invention is excellent in thermal properties and electrical properties (especially, dielectric properties).

### DESCRIPTION OF THE EMBODIMENTS

Hereinafter, embodiments of the present invention will be described in detail, but the present invention is not limited to these embodiments.

The compound having unsaturated double bonds according to the present invention (hereinafter, also simply referred to as “the compound of the present invention”) is represented by the following formula (1).

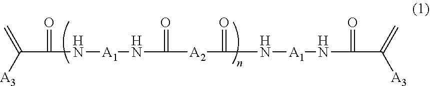

In the formula (1), A₁s each independently represent a divalent linking group represented by the following formula (1-1). (1-2), or (1-3), or a divalent linking group other than those represented by the formula (1-1), (1-2), or (1-3), provided that at least one of the two or more A₁s is a divalent linking group represented by any of the formula (1-1), (1-2), or (1-3),

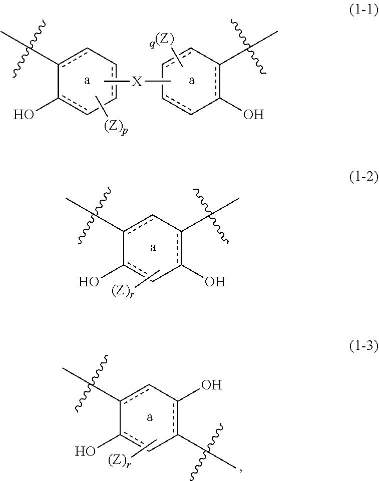

In the formulae (1-1), (1-2), and (1-3), ring a represents a benzene ring or a cyclohexane ring, X represents a direct bond or a divalent linking group (note: “direct bond” as used herein refers to an embodiment in which two rings a on both sides of X in the formula (1-1) are directly bonded without interposing an atom, a divalent linking group, or the like), Z represents a monovalent substituent of ring a, and when two or more Zs are present in the formula (1), the two or more Zs may be the same or different from each other, p, q, and r are number of substituent Z, p and q each independently represent an integer of 0 to 3, r represents an integer of 0 to 2, and when two or more ps are present in the formula (1-1), the two or more ps may be the same or different from each other, when two or more qs are present in the formula (1-1), the two or more qs may be the same or different from each other, and when two or more rs are present in the formula (1-2) or (1-3), the two or more rs may be the same or different from each other.

When two or more divalent linking groups represented by the formula (1-1), (1-2), or (1-3) are present in the formula (1), all of the two or more rings a present in the formula (I) may be benzene rings, all of them may be cyclohexane rings, or alternatively, both a benzene ring(s) and a cyclohexane ring(s) are present together. It is preferred that all of the two or more rings a are benzene rings.

The divalent linking group represented by X in the formula (1-1) is not particularly limited, as long as it is the divalent linking group of a publicly known diamine compound in which two 2-aminophenol compounds optionally having a substituent are bonded through a divalent linking group or a publicly known diamine compound in which two 2-aminocyclohexanol compounds optionally having a substituent are bonded through a divalent linking group. Examples for the divalent linking group in the diamine compound include the divalent linking group of 3,3′-diamino-4,4′-dihydroxydiphenyl ether which is an oxygen atom, and the divalent linking group of 3,3′-diamino-4,4′-dihydroxydiphenylmethane which is a methylene group.

The divalent linking group represented by X in the formula (1-1) is preferably a divalent linking group represented by any one of the following formulae (a) to (f), from the viewpoint of the solubility of a compound having unsaturated double bonds finally obtained or film properties of a cured product of a composition containing the compound having unsaturated double bonds (described later). Another preferred example of the divalent linking group represented by X may be —O-Ph-O—. More preferred group is a divalent linking group represented by any one of the following formulae (c) and (d).

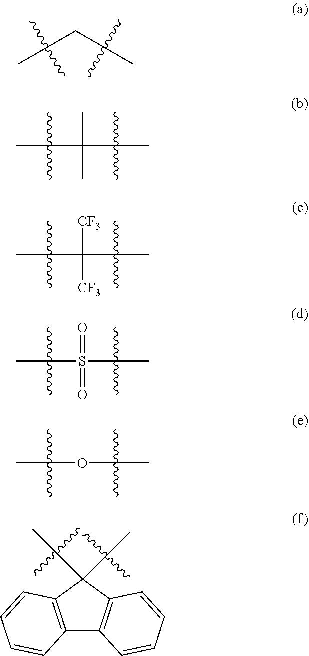

X in the formula (1-1) is preferably a direct bond or a divalent linking group containing one or more selected from the group consisting of a carbon atom, a fluorine atom, a sulfur atom, and an oxygen atom. A direct bond or a divalent linking group represented by any one of the formulae (a) to (f) is more preferred, and a direct bond or a divalent linking group represented by the formula (c) or (d) is still more preferred.

The monovalent substituent represented by Z in the formulae (1-1), (1-2) and (1-3) is not particularly limited, but is preferably a halogen atom, a hydroxyl group, a nitro group, a cyano group, an aliphatic group, an aromatic group, an acetyl group, a carboxy group, an ester group, an amide group, a trifluoromethyl group, an imide group, or a urea group. When two or more Zs are present, each Z may be the same or different from each other.

Specific examples for the halogen atom which is a preferred embodiment of Z in the formulae (1-1), (1-2) and (1-3) may include a fluorine atom, a bromine atom, a chlorine atom, and an iodine atom. A fluorine atom is preferred.

The aliphatic group which is a preferred embodiment of Z in the formulae (1-1), (1-2) and (1-3) is a residue obtained by removing one hydrogen atom from a hydrocarbon compound having no aromaticity. The hydrocarbon compound is not limited to ones having a linear chain, a branched chain, or a cyclic chain, and may be a compound having two or more of these chains.

Specific examples of the aliphatic group which is a preferred embodiment of Z in the formulae (1-1), (1-2), and (1-3) may include alkyl groups having 1 to 6 carbon atoms such as a methyl group, an ethyl group, a propyl group, and a cyclohexane group, alkenyl groups having 1 to 6 carbon atoms such as propene, alkynyl groups having 1 to 6 carbon atoms such as propyne, etc. A methyl group is preferred.

The aromatic group which is a preferred embodiment of Z in the formulae (1-1), (1-2), and (1-3) refers to a residue obtained by removing one hydrogen atom from the aromatic ring of an aromatic compound. The aromatic compound is not limited to any of an aromatic hydrocarbon compound, an aromatic heterocyclic compound, a heterocyclic condensed aromatic compound, and the like, as long as it is a compound having aromaticity.

Specific examples for the aromatic group which is a preferred embodiment of Z in the formulae (1-1), (1-2), and (1-3) may include a phenyl group, a naphthyl group, etc. A phenyl group is preferred.

Specific examples for the ester group which is a preferred embodiment of Z in the formulae (1-1), (1-2), and (1-3) may include a methoxycarbonyl group, an ethoxycarbonyl group, an n-propoxycarbonyl group, a benzyloxycarbonyl group, and a phenoxycarbonyl group. A phenoxycarbonyl group is preferred.

Specific examples for the amide group which is a preferred embodiment of Z in the formulae (1-1), (1-2), and (1-3) may include alkylamide groups such as —CONH₂, —CONH(CH₃), and —CONH(i-C3H7), a benzamide group, a naphthamide group, a p-t-butylbenzamide group, an o-chlorobenzamide group, an arylamide group such as —CON(Ph)₂, etc. A benzamide group is preferred. In the present specification, Ph represents a phenyl group.

Z in the formulae (1-1), (1-2), and (1-3) is more preferably an aliphatic group from the viewpoint of the ease of intramolecular dehydration ring closure of the polymer at low temperatures.

It is preferred that p, q, and r in the formulae (1-1), (1-2), and (1-3) are each independently an integer of 0 or 1.

The phenolic hydroxyl group of the formulae (1-1), (1-2), and (1-3) can be converted to another substituent via an oxygen atom of the hydroxyl group, but preferably remains as a phenolic hydroxyl group. More preferably 50% or more of the phenolic hydroxyl group remains as a phenolic hydroxyl group, still more preferably 80% or more of the phenolic hydroxyl group remains as a phenolic hydroxyl group, and most preferably, 100% of the phenolic hydroxyl group remains as a phenolic hydroxyl group. Having remaining phenolic hydroxyl group can allow the development with an alkaline aqueous solution and a ring-closing reaction by the curing in the final stage, so that the strength of the cured film can be secured without deteriorating the electrical properties.

The divalent linking group represented by A₁ in the formula (1) other than the formulae (1-1), (1-2), and (1-3) is not particularly limited, as long as it is a divalent linking group obtained by removing two amino groups from a diamine compound having no phenolic hydroxyl groups (with the exception of hydrazine). Examples for the diamine compound which can serve as the divalent linking group may include aliphatic diamines having no phenolic hydroxyl groups and aromatic diamines having no phenolic hydroxyl groups, etc. Preferred are linear aliphatic diamines such as ethylenediamine, 1,3-propandiamine, and 1,4-butanediamine, aliphatic diamines having an alicyclic structure such as norbomanediamine and 1,3-bisaminomethylcyclohexane, and aliphatic diamines having a branched structure such as dimer diamine. A dimer diamine is preferred from the viewpoint of the solubility of the compound having unsaturated double bonds finally obtained as well as from the viewpoint of the film properties of the cured film of the composition containing the compound having unsaturated double bonds.

Dimer diamine refers to a compound obtained by substituting two primary amino groups for the two carboxy groups of a dimer acid which is a dimer of unsaturated fatty acid such as oleic acid (see, for example, JPH9-12712A). Specific examples for commercially available products of such a dimer diamine may include Priamine 1074 and Priamine 1075 (both manufactured by Croda Japan K.K.), and Versamine 551 (manufactured by Cognis Japan K.K.), etc. These may be used alone or in combination of two or more. Shown below are non-limiting general formulae of dimer diamine. (In each formula, m+n is preferably 6 to 17, p+q is preferably 8 to 19, and the broken line means a carbon-carbon single bond or a carbon-carbon double bond.)

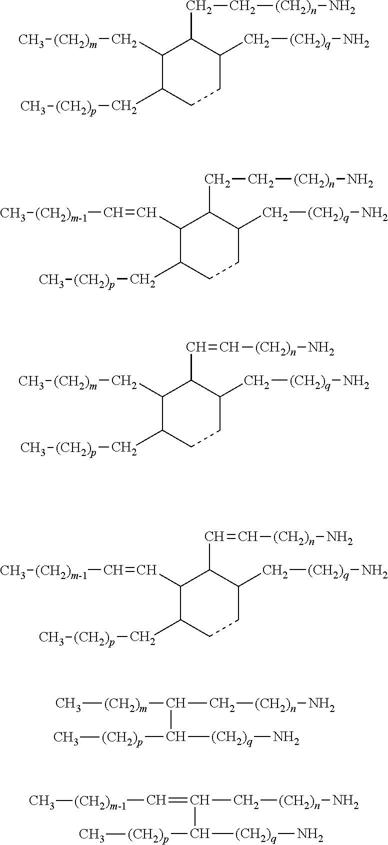

When a direct bond or a divalent linking group obtained by removing two amino groups from a diamine compound having no phenolic hydroxyl groups is introduced into Aⱼ in the formula (1) as a divalent linking group other than the formulae (1-1), (1-2), and (1-3), the introduction amount of the direct bond or the divalent linking group derived from the diamine compound having no phenolic hydroxyl groups is preferably 30% or less of all A₁s. This preference is made from the viewpoint of the solubility of the finally obtained compound having unsaturated double bonds as well as from the viewpoint of the film properties of the cured film of the composition containing the compound having unsaturated double bonds. Incidentally, the term “direct bond” as used herein refers to an aspect in which two —NH— groups clearly indicated on both sides of A₁ in the formula (1) are directly bonded without interposing an atom, a divalent linking group, or the like. The phrase “30% or less of all A₁s” as used herein means that, for example, when the compound of the present invention has 10 A₁ units, the number (average) of the direct bond and the divalent linking groups derived from a diamine compound, having no phenolic hydroxyl groups is 1 to 3. It is possible to control the introduction amount of the direct bond and the divalent linking group derived from a diamine compound by changing the content (mol %) of each of hydrazine and the diamine compound having no phenolic hydroxyl groups contained in the diamine component to be used in synthesizing the compound of the present invention.

It is preferred that in the formula (1) all of A₁s are divalent linking groups selected from the group consisting of formulae (1-1), (1-2), and (1-3), or at least one of A₁s is a divalent linking group obtained by removing two amino groups from an aliphatic diamine compound, and the rest of A₁s is(are) a divalent linking group(s) selected from the group consisting of the formulae (1-1), (1-2), and (1-3). When all of A₁s in the formula (1) are divalent linking groups of the formula (1-1), the cured film of the composition containing the compound having unsaturated double bonds is excellent in flexibility.

In the formula (1), A₂ represents a divalent linking group obtained by removing two carboxy groups from a saturated aliphatic dicarboxylic acid compound, a divalent linking group obtained by removing two carboxy groups from an aromatic dicarboxylic acid compound, or a divalent linking group other than a divalent linking group obtained by removing two carboxy groups from a saturated aliphatic dicarboxylic acid compound or a divalent linking group obtained by removing two carboxy groups from an aromatic dicarboxylic acid compound (hereinafter simply referred to as “other divalent linking group”), and when two or more A₂s are present, the two or more A₂s may be the same or different from each other, A₂ is preferably a divalent linking group obtained by removing two carboxy groups from a saturated aliphatic dicarboxylic acid compound, and when n=1 in the formula (1), A₂ is preferably a divalent linking group obtained by removing two carboxy groups from a saturated aliphatic dicarboxylic acid compound, and when n>1 in the formula (1), it is preferred that at least one of two or more A₂s is a divalent linking group obtained by removing two carboxy groups from a saturated aliphatic dicarboxylic acid compound. Furthermore, it is the most preferred that all A₂s are a divalent linking group obtained by removing two carboxy groups from a saturated aliphatic dicarboxylic acid compound. When at least one of A₂(s) is a divalent linking group obtained by removing two carboxy groups from a saturated aliphatic dicarboxylic acid compound, it is possible to make the compound having unsaturated double bonds excellent in electrical properties.

When at least one of A₂(s) in the formula (1) is a divalent linking group obtained by removing two carboxy groups from a saturated aliphatic dicarboxylic acid compound, it is possible to make the compound excellent in electrical properties. Among the electrical properties, the dielectric constant (Dk) is preferably 2.7 or less, more preferably 2.6 or less, and still more preferably 2.5 or less. The dielectric dissipation factor (Df) is preferably 0.010 or less, more preferably 0.008 or less, still more preferably 0.006 or less, and further more preferably 0.004 or less. Transmission loss can be suppressed by reducing Dk and Df.

Incidentally, the dielectric constant (Dk) and the dielectric dissipation factor (Df) used herein refer to values measured at a frequency of 10 GHz and a temperature of 25° C. by means of a cavity resonator perturbation method as will be described in Examples later.

Examples for the saturated aliphatic dicarboxylic acid compound for A₂ include saturated alicyclic dicarboxylic acid such as malonic acid, dimethyl malonic acid, ethyl malonic acid, isopropylmalonic acid, di-n-butylmalonic acid, succinic acid, tetrafluorosuccinic acid, methyl succinic acid, 2,2-dimethyl succinic acid, 2,3-dimethyl succinic acid, glutaric acid, hexafluoroglutaric acid, 2-methylglutaric acid, 3-methylglutaric acid, 2,2-dimethylglutaric acid, 3,3-dimethylglutaric acid, 3-ethyl-3 methylglutaric acid, adipic acid, octafluoroadipic acid, 3-methyladipic acid, pimelic acid, 2,2,6,6-tetramethylpimelic acid, suberic acid, dodecafluorosuberic acid, azelaic acid, sebacic acid, hexafluorosebacic acid, 1,9-nonanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, pentacosanedioic acid, hexacosanedioic acid, heptacosanedioic acid, octacosanedioic acid, nonacosanedioic acid, triacontandioic acid, hentriacontandioic acid, dotriacontandioic acid, diglycolic acid, and cyclohexane-1,4-dicarboxylic acid (cis/trans/cis and trans mixtures), but are not limited thereto. The aliphatic chain may partially contain a hetero atom such as an oxygen atom or a sulfur atom.

Among them, from the viewpoint of the mechanical properties of the cured film, it is preferred that average number of carbon atoms of the divalent saturated aliphatic chain obtained by removing two carboxy groups from a saturated aliphatic dicarboxylic acid compound for A₂ is 2 or more, and more preferably 4 or more. The average number of carbon atoms is preferably 20 or less, and more preferably 10 or less. The average number of carbon atoms is more preferably 2 or more and 20 or less or 4 or more and 20 or less, still more preferably 2 or more and 10 or less or 4 or more and 10 or less.

Having a saturated aliphatic chain with a carbon number of 2 or more or 4 or more in A₂ can allow a cured film with sufficient mechanical properties to be obtained. Having a saturated aliphatic chain with a carbon number of 20 or less or 10 or less can allow the securing of good compatibility between the compound and other components and good photosensitivity of the polybenzoxazole formed from a polymer of the compound.

In the formula (1), when n>1, two or more A₂s may each independently be a divalent linking group obtained by removing two carboxy groups from different saturated aliphatic dicarboxylic acid compounds. Alternatively, in the formula (1), when n>1, all of the two or more A₂s may be a divalent linking group obtained by removing two carboxy groups from a same saturated aliphatic dicarboxylic acid compound. In any of these cases, the preferred number of carbon atoms is the same as described above.

Examples for the aromatic dicarboxylic acid compound include phthalic acid, isophthalic acid, 1,4-benzenedicarboxylic acid, 4,4′-dicarboxydiphenyl ether(4,4′-oxybisbenzoic acid), 4,4′-dicarboxydiphenylsulfone, 4,4′-biphenyldicarboxylic acid, benzophenone-4,4′-dicarboxylic acid, and 4,4′-stilbenedicarboxylic acid, but are not limited thereto. Among them, isophthalic acid and 4,4′-dicarboxydiphenyl ether are preferred because they are exhibit excellent physical properties of the cured film or excellent solubility when used as a divalent linking group obtained by removing two carboxy groups. These compounds can be used alone or in combination of two or more. By introducing a divalent linking group obtained by removing two carboxy groups from an aromatic dicarboxylic acid compound as A₂, it is possible to improve physical properties such as thermal properties and electrical properties of the cured film of the composition containing the compound having unsaturated double bonds represented by the formula (1).

The dicarboxylic acid compound that can serve as the other divalent linking group is not particularly limited, as long as it is a dicarboxylic acid compound other than a saturated aliphatic dicarboxylic acid compound or an aromatic dicarboxylic acid compound. Examples thereof include unsaturated aliphatic dicarboxylic acid compounds. Examples of the unsaturated aliphatic dicarboxylic acid compounds that can serve as the other divalent linking group include, but not limited to, methylene succinic acid, allyl malonic acid, isopropylidene succinic acid, 2.4-hexadiene diacid, and acetylenedicarboxylic acid.

A₃ may be a hydrogen atom, a methyl group, or a combination thereof. In general, methyl group is preferred because methyl group can result in the formation of a cured film excellent in transparency and low hygroscopicity.

In the compound having unsaturated double bonds represented by the formula (1), average repetition number (n) of a constituent unit(s) composed of a combination of a diamine-derived moiety and a dicarboxylic acid-derived moiety is 1≤n≤9, preferably 1.0≤n≤5.0, and more preferably 1.0<n≤4.0.

When the average repetition number (n) is 1 or more, a compound having the unsaturated double bond of the present invention is excellent in film formability, and it is possible to suppress generation of cracks (cracking on composition layer) that may occur when a composition containing the compound is irradiated with a light, by which an appropriate cured film structure can be obtained. When the average repetition number (n) is 9 or less, the proportion of the unsaturated double bonds in the compound molecule can be moderately secured, resulting in excellent curability. The average repetition number (n) as used herein refers to a value calculated from molar ratios of raw materials charged into the reaction system for synthesis of the compound. For example, when a charged molar amount of a raw material diamine is 2.0 times a charged molar amount of a raw material dicarboxylic acid compound, the average repetition number (n) can calculated to be 1.0.

The method of producing the compound of the present invention is not particularly limited, but generally may be a method in which a diamine compound including a diaminodiphenol compound or a cyclohexane ring compound obtained by hydrogenating the benzene ring of a diaminodiphenol compound (hereinafter referred to as a diaminodiphenol compound or a hydrogenated product thereof) and a dihalide derivative of a dicarboxylic acid compound including a saturated aliphatic dicarboxylic acid compound or an aromatic dicarboxylic acid compound (and, as an optional component, another dicarboxylic acid compound, e.g., an unsaturated aliphatic dicarboxylic acid compound) are subjected to a dehydrochlorination reaction with an excess mole of the diamine compound (with an average repetition number (n) is 1≤n≤9, preferably 1.0<n≤5.0, more preferably 1.0≤n≤4.0) thereby obtaining a dehydrochlorination reaction product; and thereafter, a halide derivative of acrylic acid or methacrylic acid is reacted with the amino groups of the dehydrochlorination reactant obtained above at both terminals. Alternatively, in this general production method, it is possible to replace the diaminodiphenol compound with a diaminocatechol compound (i.e., a raw material for production corresponding to the compound of the formula (1) with A₁ represented by the formula (1-2) or (1-3)) (the same applies to the following description of the production method). Halide derivatives of acrylic acid or methacrylic acid (hereinafter referred to as “(meth)acrylic acid”) can be used alone or in combination.

When a diamine having no phenolic hydroxyl groups is used in combination in the reaction, the procedure of the reaction is not particularly limited, as long as diamine compounds used in the reaction have a total molar amount greater than the molar amount of the dihalide derivative of the dicarboxylic acid compound, and the average repetition number (n) is 1≤n≤9. For example, it is possible to mix the diaminodiphenol compound or hydrogenated product thereof with the diamine having no phenolic hydroxyl groups in advance and then react the mixture with the dihalide derivative of a dicarboxylic acid compound, or alternatively, it is also possible to first react one of these diamine compounds with the dihalide derivative of a dicarboxylic acid compound, and then add thereto the other diamine compound for the secondary reaction.

The dihalide derivative of a dicarboxylic acid compound is preferably a dichloride derivative. Examples for a halogenating agent used in the conversion into the dichloride derivative include thionyl chloride, oxalyl chloride, phosphoryl chloride, and phosphorus chloride which can be used in an ordinary acid chlorination reaction. As the dichloride derivative of a dicarboxylic acid compound, it is possible to use a commercially available product.

As the dihalide derivative of (meth)acrylic acid to be reacted with the amino groups at both terminals of the dehydrochlorination reaction product, it is possible to use one synthesized by a publicly known method, or a commercially available product. When a synthesized one is used, (meth)acrylic acid may be halogenated using a halogenating agent.

Examples for the diaminodiphenol compound or hydrogenated product thereof used in production of the compound of the present invention include the following compounds No. 5 to 12 and cyclohexane ring compounds obtained by hydrogenating the benzene rings in compounds No. 5 to 12. These diaminodiphenol compounds or hydrogenated products thereof can be used alone or in combination of two or more.

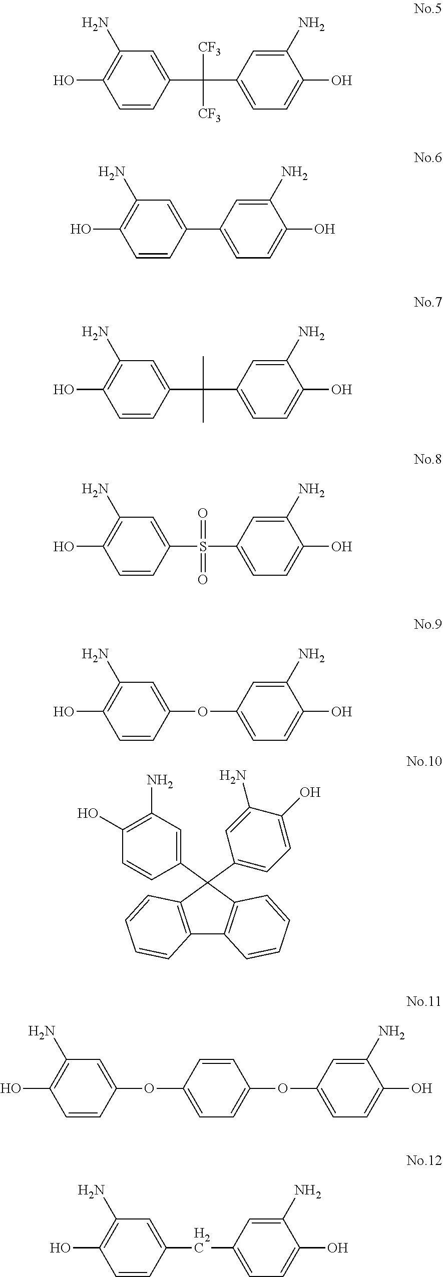

The reaction between the dihalide derivative of a dicarboxylic acid compound and a diamine compound or its hydrogenated product is desirably performed in an organic solvent in the presence of a dehalogenating agent. As the dehalogenating agent, it is usually possible to use an organic base such as pyridine, 2-methylpyridine, or triethylamine. As the organic solvent, sulfolane, N, N-dimethylacetamide, N-methyl-2 pyrrolidone, N, N-dimethylformamide, or the like can be used. The concentration of the reaction components with respect to the entire reaction system including the solvent is not particularly limited, but is preferably 10 to 50 mass %, and more preferably 20 to 40 mass %.

As a specific synthesis procedure, a diamine compound is dissolved in an organic solvent, and the dihalide derivative of a dicarboxylic acid compound is added thereto. The temperature at which the dihalide derivative of a dicarboxylic acid compound is added to the reaction system may be preferably −20 to 35° C., and more preferably −10 to 30° C. The reaction temperature of a diamine compound and the dihalide derivative of a dicarboxylic acid compound may be preferably 0 to 80° C., and more preferably 10 to 40° C. The reaction time may be preferably 30 minutes to 24 hours, and more preferably 1 to 5 hours. A halide derivative of (meth)acrylic acid is then added to the reaction system. The temperature at which the halide derivative of (meth)acrylic acid is added to the reaction system may be preferably −20 to 20° C., and more preferably −10 to 10° C. After the addition of the halide derivative of (meth)acrylic acid, the reaction temperature may be preferably 0 to 30° C., and more preferably 0 to 10° C.; and the reaction time may be preferably 10 minutes to 3 hours, and more preferably 30 minutes to 2 hours. After completion of the reaction, water is charged into the obtained reaction liquid, by which the compound having unsaturated double bonds according to the present invention can be obtained.

It is possible to remove impurities from the compound obtained by the above procedure, by using an acidic aqueous solution, an alkaline aqueous solution, a neutral aqueous solution, an organic solvent, or the like.

The above description relates to a method of producing the compound of the present invention using a diamine compound including a diaminodiphenol compound or a hydrogenated product thereof, a dihalide derivative of a dicarboxylic acid compound, and a halide derivative of (meth)acrylic acid. Alternatively, the compound of the present invention may be produced by a known dehydration condensation reaction using a diaminodiphenol compound or a hydrogenated product thereof, a dicarboxylic acid compound, and (meth)acrylic acid.

Next, a composition of the first aspect and a composition of the second aspect according to the present invention will be described.

The composition of the first aspect of the present invention contains at least one kind of the compound having unsaturated double bonds of the present invention represented by the formula (1), and at least one kind of photopolymerization initiator or at least one kind of curing catalyst.

The photopolymerization initiator that can be used in the composition of the first aspect of the present invention is not particularly limited, as long as it is a conventionally publicly known photopolymerization initiator which has been used for photocuring a compound having an unsaturated double bond group. Specific examples for the photopolymerization initiator include acetophenone, 2,2-dimethoxyacetophenone, p-dimethylaminoacetophenone, Michler's ketone, benzyl, benzoin, benzoin methyl ether, benzoin ethyl ether, benzoin isopropyl ether, benzoin n-propyl ether, benzoin isobutyl ether, benzoin n-butyl ether, benzyl dimethyl ketal, thioxanthone, 2-chlorothioxanthone, 2-methylthioxanthone, 2,2-dimethoxy-1,2-diphenylethane-1-one, 1-hydroxy-cyclohexyl-phenyl-ketone, 2-hydroxy-2-methyl-1-phenyl-propane-1-one, 1-[4-(2-hydroxyethoxy)-phenyl]-2-hydroxy-2-methyl-1-propane-1-one, 2-hydroxy-1-{4-[4-(2-hydroxy-2-methyl-propionyl)-benzyl]phenyl}-2-methyl-propane-1-one, 2-methyl-1-[4-(methylthio)phenyl]-2-morpholinopropane-1-one, 2-benzyl-2-dimethylamino-1-(4-morpholinophenyl)-1-butanone, 2,4,6-trimethylbenzoyl-diphenyl-phosphine oxide, bis(2,4,6-trimethylbenzoyl)-phenylphosphine oxide, 1-[4-(phenylthio) phenyl]-1,2-octanedione=2-(O-benzoyloxime); ethanone, 1-[9-ethyl-6-(2-methylbenzoyl)-9H-carbazole-3yl]-,1-(O-acetyloxime); 2,4-diethylthioxanthone, etc. These photopolymerization initiators may be used alone or in combination of two or more.

Among them, it is preferred to use a photopolymerization initiator capable of efficiently generating radicals at an exposure wavelength of 310 to 436 nm (more preferably 365 nm), from the viewpoint that it enables the formation of fine patterns by a reduction projection exposure machine (Stepper, light source wavelength: 365 nm, 436 nm) that is typically used in a process of manufacturing semiconductor protective films or the like. Preferred examples for the photopolymerization initiator include 1-[4-(phenylthio)phenyl]-1,2-octanedione=2-(O-benzoyloxime) (“IRGACURE OXE-01” manufactured by BASF Japan Ltd.); ethanone, 1-[9-ethyl-6-(2-methylbenzoyl)-9H-carbazole-3yl]-, 1-(O-acetyloxime) (“IRGACURE OXE-02” manufactured by BASF Japan Ltd.); 2,4-diethylthioxanthone (“DETX-S” manufactured by Nippon Kayaku Co., Ltd.), and 2-methyl-1-[4-(methylthio)phenyl]-2-morpholinopropane-1-one (“Omnirad 907” manufactured by IGM Resins B.V.).

The amount of the photopolymerization initiator (when used) is preferably 0.1 to 20 parts by mass, more preferably 1 to 10 parts by mass, and still more preferably 3 to 8 parts by mass with respect to 100 parts by mass of the compound represented by the formula (1).

A sensitizer may be used in combination with the composition of the first aspect which contains a photopolymerization initiator. The sensitizer which can be used in combination is not particularly limited, as long as it is a conventionally publicly known sensitizer. Examples of the sensitizer include 4,4′-bis(diethylamino) benzophenone.

The amount of the sensitizer (when used) is preferably 2 parts by mass or less, more preferably 0.05 to 0.5 parts by mass with respect to 100 parts by mass of the compound represented by the formula (1). The combined use of the sensitizer allows the enhancement of sensitivity to light during a self-polymerization reaction.

The curing catalyst used in the composition according to the first aspect of the present invention is not particularly limited, as long as it allows the self-polymerization reaction of an acryloyl group or a methacryloyl group (hereinafter referred to as a “meth(acryloyl) group”) of the compound of the formula (1) according to the present invention at both terminals to be promoted by heating. Those conventionally used can appropriately be employed. Specific examples of the curing catalyst include imidazoles such as 2-methylimidazole, 2-ethylimidazole, 2-phenylimidazole, 2-ethyl-4 methylimidazole, 2-undecylimidazole, and 1-cyanoethyl-2-ethyl-4-methylimidazole; amines such as triethylamine, triethylenediamine, 2-(dimethylaminomethyl) phenol, 1,8-diaza-bicyclo(5,4,0) undecene-7, tris(dimethylaminomethyl) phenol, and benzyldimethylamine; phosphines such as triphenylphosphine, tributylphosphine, and trioctylphosphine; organic metal salts such as tin octylate, zinc octylate, dibutyltin dimaleate, zinc naphthenate, cobalt naphthenate, and tin oleate; and metal chlorides such as zinc chloride, aluminum chloride, and tin chloride, organic peroxides such as di-tert-butyl peroxide and dicumyl peroxide, azo compounds such as azobisisobutyronitrile and azobisdimethylvaleronitrile, mineral acids such as hydrochloric acid, sulfuric acid and phosphoric acid, Lewis acids such as boron trifluoride, and salts such as sodium carbonate and lithium chloride.

The amount of the curing catalyst (when used) is preferably 10 parts by mass or less, more preferably 1 to 5 parts by mass with respect to 100 parts by mass of the compound represented by the formula (1).

The composition of the second aspect of the present invention contains at least one of the compound having unsaturated double bonds of the present invention represented by the formula (1) and at least one of compounds capable of reacting with an unsaturated double bond (compounds capable of reacting with a meth(acryloyl) group).

Examples of the compound capable of reacting with an unsaturated double bond used in the composition of the second aspect of the present invention include compounds having an unsaturated double bond other than the compound having unsaturated double bonds of the present invention represented by the formula (1).

Examples of the compound having an unsaturated double bond other than the compound having unsaturated double bonds of the present invention represented by the formula (1) include compounds having at least one of an acrylic group, a methacrylic group, an allyl group, a styryl group, and the like.

Specific examples of a compound having two or more acrylic groups which can be used as the compound capable of reacting with an unsaturated double bond include hydrogenated dicyclopentadienyl diacrylate, dicyclopentenyl acrylate, dicyclopentenyloxyethyl acrylate, 1,3-butanediol diacrylate, 1,4-butanediol diacrylate, 1,6-butanediol diacrylate, diethylene glycol diacrylate, neopentyl glycol diacrylate, polyethylene glycol 200 diacrylate, polyethylene glycol 400 diacrylate, polyethylene glycol 600 diacrylate, diethylene glycol diacrylate, neopentyl glycol diacrylate, hydroxypivalinic acid ester neopentyl glycol diacrylate, triethylene glycol diacrylate, bis(acryloxyethoxy) bisphenol A, bis(acryloxyethoxy) tetrabromobisphenol A, tripropylene glycol diacrylate, trimethylolpropane triacrylate, pentaerythritol triacrylate, pentaerythritol tetraacrylate, dipentaerythritol hexaacrylate, and dipentaerythritol monohydroxypentaacrylate.

Specific examples of the compound having two or more methacrylic groups that can be used as the compound capable of reacting with an unsaturated double bond include a compound group in which the acrylic groups in the compound having two or more acrylic groups are replaced with methacrylic groups.

Specific examples of the compound having two or more allyl groups that can be used as the compound capable of reacting with an unsaturated double bond include diallyl adipate, diallyl fumarate, diallyl hexahydrophthalate, pentaerythritol tetraallyl ether, glycerol diallyl ether, triallyl citrate, diallyl isophthalate, and diallyl phthalate.

Specific examples of the compound having two or more styryl groups that can be used as the compound capable of reacting with an unsaturated double bond include bis(vinylphenyl) methane, bis(vinylphenyl) ethane, and bis(vinylphenyl) hexane, etc.

Other examples of the compound capable of reacting with an unsaturated double bond include a compound having a functional group containing an unsaturated double bond therein such as styrene, divinylbenzene, or a maleimide group, etc.

The compound capable of reacting with an unsaturated double bond is not limited to the examples listed here.

In the composition of the first aspect or the second aspect of the present invention, it is possible to use another component in combination, in addition to the compound having unsaturated double bonds represented by the formula (1) that is an essential component, and an optional photopolymerization initiator and/or curing catalyst, or an optional compound capable of reacting with an unsaturated double bond (i.e., a compound capable of reacting with a meth(acryloyl) group).

Examples of another component which can be used in combination with the composition of the first aspect or the second aspect of the present invention include various additives such as an organic solvent, an adhesion enhancer such as a coupling agent, a thermoplastic resin, a colorant, a thickener, a thermal polymerization inhibitor, an antifoaming agent, and a leveling agent.

Examples of the organic solvent include, but not limited to, γ-butyrolactone, ethyl lactate, propylene glycol monomethyl ether acetate, benzyl acetate, n-butyl acetate, ethoxyethyl propionate, 3-methyl methoxypropionate, N-methyl-2-pyrrolidone, N-ethyl-2-pyrrolidone, N,N-dimethylformamide. N,N-dimethylacetamide, 4-formylmorpholine, 1,3-dimethyl-2-imidazolidinone, dimethyl sulfoxide, hexamethylphosphorylamide, tetramethylene sulfone, cyclohexanone, cyclopentanone, diethyl ketone, diisobutyl ketone, and methyl amyl ketone. These organic solvents can be used alone or in combination of two or more. The combined use of organic solvents is a preferred embodiment from the viewpoint that it can improve handling of the composition.

The amount of the organic solvent in the composition of the first aspect or the second aspect of the present invention (when used) may be usually 95 mass % or less and preferably 20 to 90 mass % relative to the total mass of the composition, but it is not limited thereto.

The addition of a compound containing a tertiary amine structure as an organic solvent can promote a reaction of the meth(acryloyl) group. Therefore, among the examples of organic solvent listed above, it is desirable to use N-methyl-2-pyrrolidone. N-ethyl-2-pyrrolidone, N, N-dimethylformamide, N, N-dimethylacetamide, 4-formylmorpholine, or 1,3-dimethyl-2-imidazolidinone alone as a compound containing a tertiary amine structure, or in combination with a compound which does not contain a tertiary amine structure.

When a compound containing a tertiary amine structure and a compound not containing a tertiary amine structure are used in combination, the ratio of their amounts is not particularly limited. It is preferred that the amount of the compound containing a tertiary amine structure is 50% or less relative to the total amount of a solvent, because the compound containing a tertiary amine structure generally has a high boiling point, which may require a long drying time after a spin coating step.

The coupling agent that can be used as the adhesion enhancer is not particularly limited, but it may typically be a silane coupling agent. Examples of the silane coupling agent include, but not limited to, 3-chloropropyltrimethoxysilane, vinyltrichlorosilane, vinyltriethoxysilane, vinyltrimethoxysilane, vinyltris(2-methoxyethoxy) silane, 3-methacryloxypropyltrimethoxysilane, 2-(3,4-epoxycyclohexyl) ethyltrimethoxysilane, 3-glycidoxypropyltrimethoxysilane, 3-mercaptopropyltrimethoxysilane, 3-aminopropyltriethoxysilane, N-2-(aminoethyl)-3-aminopropyltrimethoxysilane, and 3-ureidopropyltriethoxysilane. These can be used alone or in combination of two or more.

The silane coupling agent is uncapable of reacting with the compound having unsaturated double bonds of the present invention, or the like (i.e., the compound itself, a self-polymer of the compound, a copolymer with a compound capable of reacting with an unsaturated double bond, or a polybenzoxazole). Hence, a remaining portion of the silane coupling agent other than a portion of the silane coupling agent acting on the interface between a substrate and the cured product can be present as a residue after the curing reaction. Therefore, the silane coupling agent as an adhesion enhancer may give an undesirable influence such as deterioration of physical properties, when it is used in a large amount. It is appropriate to use the silane coupling agent in an amount controlled so as not to give an undesirable influence, because it can exhibit the desired enhance effect of adhesion even in a small amount depending on the type of a substrate to be applied. The amount of the silane coupling agent (when used) is usually 15 mass % or less, preferably more than 0 mass % and 5 mass % or less with respect to the total mass of the composition, but the upper limit of the amount can vary depending on the type of a substrate.

Examples of the thermoplastic resin include polyethersulfone, polystyrene, and polycarbonate.

Examples of a colorant include phthalocyanine blue, phthalocyanine green, iodine green, crystal violet, titanium oxide, carbon black, and naphthalene black.

Examples of the thickener include orbene, bentone, and montmorillonite.

Examples of for the thermal polymerization inhibitor may include hydroquinone, 2,6-di-tert-butyl-p-methylphenol, etc.

Examples of the antifoaming agent include silicone-based, fluorine-based, and polymer-based antifoaming agents.

The amount of each of these additives (when used) is, for example, preferably 30 mass % or less in the composition of the first aspect or the second aspect of the present invention as a guide, but may be appropriately increased or decreased according to a purpose of use.

In the composition of the first aspect or the second aspect of the present invention, for example, an inorganic filler such as barium sulfate, barium titanate, silicon oxide, amorphous silica, talc, clay, magnesium carbonate, calcium carbonate, aluminum oxide, aluminum hydroxide, or mica powder may be used in combination. The amount of the inorganic filler (when used) is preferably 60 mass % or less in the composition of the first aspect or the second aspect of the present invention.

The composition of the first aspect or the second aspect of the present invention may contain a thermal acid generator (thermal latent acid generator) or a thermal base generator (thermal latent base generator). It is preferred to use such a thermal acid generator or thermal base generator, because it can efficiently act as a catalyst when a polymer of a phenolic hydroxyl group-containing polyamide structure (or, a nonphenolic hydroxyl group-containing polyamide structure in the case where the benzene ring is hydrogenated to form the cyclohexane ring), which is a polybenzoxazole precursor formed from the compound having unsaturated double bonds of the formula (1), causes an intramolecular dehydration ring-closing reaction for cyclization. In particular, an acid generated from a thermal acid generator is capable of lowering the temperature of the intramolecular dehydration ring-closing reaction, and a cured film obtained therefrom has a performance comparable to that of a film cured at high temperatures.

The acid generated from the thermal acid generator is preferably a strong acid. Preferably, specific examples of such a strong acid include arylsulfonic acids such as p-toluenesulfonic acid and benzenesulfonic acid, and alkylsulfonic acids such as camphorsulfonic acid, trifluoromethylsulfonic acid, and nonafluorobutanesulfonic acid. These acids can efficiently act as a catalyst when a polymer of a phenolic hydroxyl group-containing polyamide structure (or, a nonphenolic hydroxyl group-containing polyamide structure in the case where the benzene ring is hydrogenated to form the cyclohexane ring), which is a polybenzoxazole precursor formed from the compound having unsaturated double bonds of the formula (1), causes an intramolecular dehydration ring-closing reaction for cyclization.

It is also possible to use the compound of the present invention and the composition of the first aspect or the second aspect comprising the compound in the form of a dry film resist. That is, it is possible to form a dry film resist by applying the compound of the present invention and the composition of the first aspect or the second aspect onto a base film using a roll coater, a die coater, a knife coater, a bar coater, a gravure coater, or the like, drying the coat in a drying furnace set at 45 to 140° C. to remove a predetermined amount of solvent, and, as necessary, laminating a cover film or the like on the coat. At this time, the thickness of the resist on the base film can be controlled in the range of from 2 to 200 μm. The compound of the present invention and the composition of the first aspect or the second aspect used for forming the dry film resist may be any of those described above, as long as they are ones containing the compound. Examples of the base film and the cover film include films of polyester, polypropylene, polyethylene, TAC, polyimide, etc. As these films, it is possible to use a film treated with a silicone-based release treatment agent or a non-silicone-based release treatment agent, as necessary. Supplying the compound and the composition as a dry film resist, it is possible to omit the steps of coating the composition on a support and drying the coat, by which the composition of the first aspect or the second aspect of the present invention can be used in a simpler manner.

The polybenzoxazole of the present invention is an intramolecular dehydrated ring-closed product of a polymer formed from the compound having unsaturated double bonds of the present invention described above (i.e., a self-polymer of the compound or a copolymer of the compound and a compound capable of reacting with an unsaturated double bond).

In the entire specification and appended claims, not only when the ring a is a benzene ring, but also when the ring a is a cyclohexane ring formed by hydrogenation of the benzene ring, the intramolecular dehydrated ring-closed products of polymers formed from the compound having unsaturated double bonds of the present invention are collectively referred to as “polybenzoxazole” for convenience.

The conditions of the polymerization reaction for forming a polymer from a compound having unsaturated double bonds according to the present invention and the conditions for intramolecular dehydration ring-closing of a polymer formed from the compound of the present invention are not particularly limited, as long as they are conditions that can be usually used in a polymerization reaction of a compound comprising an unsaturated double bond and conditions that can be usually used in an intramolecular dehydration ring-closing reaction to convert a polybenzoxazole precursor into a polybenzoxazole, respectively. However, when a polymer which is a polybenzoxazole precursor formed from the compound having unsaturated double bonds of the present invention is converted into a polybenzoxazole by an intramolecular dehydration ring-closing reaction, it is advantageous that the reaction can be successfully progressed at a considerably lower temperature (at 225° C. or lower) than usual.

The conditions of the polymerization reaction for forming a polymer from the compound having unsaturated double bonds of the present invention may be set as follows, but this is a non-limiting example. For example, the composition of the first aspect or the second aspect of the present invention may be applied to a silicon substrate and then dried at 80 to 110° C. for 3 to 10 minutes to form a composition layer having a thickness of about 5 to 15 μm on the silicon substrate, which may be then exposed to a light having a wavelength of 350 to 380 nm and heated at 130 to 170° C. for about 3 to 10 minutes.

The intramolecular dehydration ring-closing reaction can be performed using, for example, a hot plate, an oven, or a temperature raising type oven capable of setting a temperature program. The intramolecular dehydration ring-closing reaction may be performed in the air or under an atmosphere of inert gas such as nitrogen gas or argon gas.

Advantageously, the intramolecular dehydration ring-closing reaction of the polymer formed from the compound having unsaturated double bonds of the present invention can be performed at a low temperature of 225° C. or lower. The temperature of the intramolecular dehydration ring-closing reaction may preferably be lower than 225° C., and more preferably 215° C. or lower.

It is possible to use a cured film comprising the polybenzoxazole of the present invention in a semiconductor apparatus or an electronic component such as a multilayer wiring board, or in an organic EL display device. Specifically, this cured film can be suitably used in applications such as a passivation film of a semiconductor, a surface protective film of a semiconductor device, an interlayer insulating film, an interlayer insulating film of a multilayer wiring for high-density mounting, an insulating film of a rewiring layer, an interlayer insulating film of an electronic component such as an inductor or an SAW filter, and an insulating film or a flat layer of an organic electroluminescent element. The applications of the cured film are not limited thereto and can be utilized in various types of structures.

### EXAMPLES

Hereinafter, the present invention will be described more specifically with reference to Examples, but the present invention is not limited to these Examples. It is noted that “part” and “%” in the Examples are on a mass basis.

### Example 1 (Synthesis Example of Inventive Compound)

150 parts of N-methyl-2 pyrrolidone was charged into a 0.5 liter flask equipped with a stirrer and a thermometer, and 22.0 parts of 2,2′-bis(3-amino-4 hydroxyphenyl) hexafluoropropane and 15.3 parts of 2-methylpyridine were added thereto, which was followed by stirring for dissolution. While keeping the temperature of the mixture at 30° C. or lower, 12.0 parts of sebacic acid dichloride was added thereto dropwise over 15 minutes, which was followed by continuous stirring at 30° C. for 3 hours. While keeping the temperature of the obtained solution at 10° C. or lower, 2.5 parts of methacrylic acid chloride was added thereto dropwise over 10 minutes, which was followed by continuous stirring for 1 hour. 0.25 liters of water was added to the obtained solution, and a precipitate was then collected. This precipitate was dissolved in 150 parts of acetone, and the resultant solution was then dropped into 1 liter of water under stirring to recover a precipitate. Then, the precipitate was dried in an oven at 60° C. for 1 day to obtain a compound (a-1) of the present invention. The n (average repetition number) of the compound (a-1) was 5.0.

### Example 2 to 12 (Synthesis Examples of Inventive Compounds (a-2) to (a-12))

Compounds (a-2) to (a-12) according to the present invention were synthesized under the same conditions as in Synthesis Example 1 except that the raw materials and the amounts thereof were changed as shown in Table 1. When two or more raw material compounds classified into the same component were used, one of the two compounds with a smaller amount was added to the reaction system, and the other of the two compounds with a larger amount was then added thereto.

### Comparative Examples 1 to 5 (Synthesis Examples for Comparative Compounds (c-1) to (c-5))

Compounds (c-1) to (c-5) for comparison with the present invention were synthesized under the same conditions as in Synthesis Example 1 except that the raw materials and the amounts thereof were changed as shown in Table 2.

TABLE 1
Synthesis Examples

| | | Ex. 1 | Ex. 2 | Ex. 3 | Ex. 4 | Ex. 5 | Ex. 6 | Ex. 7 |
|---|---|---|---|---|---|---|---|---|
| Material | Compound name | a-1 | a-2 | a-3 | a-4 | a-5 | a-6 | a-7 |
| Diamine compound | 2,2′-bis(3-amino-4-hydroxyphenyl)hexafluoropropane | 22.0 | 23.8 | 27.5 | 33.0 | 36.6 | 27.5 | 27.5 |
| | Bis(3-amino-4-hydroxyphenyl)sulfone | | | | | | | |
| | 3,3′-diaminobiphenyl-4,4′-diol | | | | | | | |
| | 2,2′-bis(4-aminophenyl)hexafluoropropane | | | | | | | |
| Dihalide compound of | Sebacic acid dichloride | 12.0 | 12.0 | 12.0 | 12.0 | 12.0 | | |
| dicarboxylic acid | Adipic acid dichloride | | | | | | 9.2 | |
| | trans-cyclohexane-1,4-dicarboxylic | | | | | | | 10.5 |
| | acid dichloride | | | | | | | |
| | 4,4′-oxybisbenzoic acid chloride | | | | | | | |
| Terminal h [symbol] compound | Methacrylic acid chloride | 2.5 | 3. [symbol] | 6.3 | 10.0 | 12.5 | 6.3 | 6.3 |
| | Acrylic acid chloride | | | | | | | |
| Dehalogenating agent | 2-methylpyridine | 15.3 | 20.7 | 27.4 | [symbol] 7.3 | 44.0 | 27.4 | 27.4 |
| Solvent | N-methyl-2-pyrrolidone | 150.0 | 150.0 | 150.0 | 150.0 | 150.0 | 150.0 | 15 [symbol] .0 |

| | | | | | | | |
|---|---|---|---|---|---|---|---|
| n (average repetition number) | [symbol] | [symbol] | [symbol] | 1. [symbol] | 1.0 | 2.0 | 2.0 |

| | | Ex. 8 | Ex. 9 | Ex. 10 | Ex. 11 | Ex. 12 |
|---|---|---|---|---|---|---|
| Material | Compound name | a-8 | a-9 | a-10 | a-11 | a-12 |
| Diamine compound | 2,2′-bis(3-amino-4-hydroxyphenyl)hexafluoropropane | 27.5 | 27.5 | | 22.0 | 27.5 |
| | Bis(3-amino-4-hydroxyphenyl)sulfone | | | 21.0 | | |
| | 3,3′-diaminobiphenyl-4,4′-diol | | | | [symbol] .2 | |
| | 2,2′-bis(4-aminophenyl)hexafluoropropane | | | | | |
| Dihalide compound of | Sebacic acid dichloride | | 9.0 | 12.0 | 12.0 | 12.0 |
| dicarboxylic acid | Adipic acid dichloride | | | | | |
| | trans-cyclohexane-1,4-dicarboxylic | | | | | |
| | acid dichloride | | | | | |
| | 4,4′-oxybisbenzoic acid chloride | 14. [symbol] | 3. [symbol] | | | |
| Terminal h [symbol] compound | Methacrylic acid chloride | 6.3 | 6.3 | 6.3 | 6.3 | |
| | Acrylic acid chloride | | | | | 5.4 |
| Dehalogenating agent | 2-methylpyridine | 27.4 | 27.4 | 27.4 | 27.4 | 27.4 |
| Solvent | N-methyl-2-pyrrolidone | 150.0 | 150.0 | 150.0 | 150.0 | 150.0 |

| | | | | | |
|---|---|---|---|---|---|
| n (average repetition number) | 2.0 | [symbol] .0 | 2. [symbol] | [symbol] | 2.0 |

Each number [symbol] ed in the material columns represents the amount in parts by mass. [symbol]
indicates data missing or illegible when filed

TABLE 2
Comparative Synthesis Examples

| | | Comp. Ex 1 | Comp. Ex. 2 | Comp. Ex. 3 | Comp. Ex. 4 | Comp. Ex. 5 |
|---|---|---|---|---|---|---|
| Material | Compound name | c-1 | c-2 | c-3 | c-4 | c-5 |
| Diamine compound | 2,2′-bis(3-amino-4-hydroxyphenyl)hexafluoropropane | 20.1 | 54.9 | | | |
| | Bis(3-amino-4-hydroxyphenyl)sulfone | | | 15.4 | 42.0 | |
| | 3,3′-diaminobiphenyl-4,4′-diol | | | | | |
| | 2,2′-bis(4-aminophenyl)hexafluoropropane | | | | | 25.1 |
| Dihalide compound of | Sebacic acid dichloride | 12.0 | 12.0 | 12.0 | 12.0 | 12.0 |
| dicarboxylic acid | Adipic acid dichloride | | | | | |
| | trans-cyclohexane-1,4-dicarboxylic acid dichloride | | | | | |
| | 4,4′-oxybisbenzoic acid chloride | | | | | |
| Terminal halide | Methacrylic acid chloride | 1.3 | 25.1 | 1.3 | 25.1 | 6.3 |
| compound | Acrylic acid chloride | | | | | |
| Dehalogenating agent | 2-methylpyridine | 13.1 | 77.1 | 13.1 | 77.1 | 27.4 |
| Solvent | N-methyl-2-pyrrolidone | 150.0 | 200.0 | 150.0 | 200.0 | 150.0 |

| | | | | | |
|---|---|---|---|---|---|
| n (average repetition number) | 10.0 | 0.5 | 10.0 | 0.5 | 2.0 |

Each number filled in the material columns represents the amount in parts by mass.

### Preparation of Compositions of Examples 13 to 24 and Comparative Examples 6 to 10

Compositions of Examples 13 to 24 and Comparative Examples 6 to 10 were each prepared by blending the components in the amounts of the parts shown in Tables 3 and 4.

The components used in the compositions of Examples 13 to 24 and Comparative Examples 6 to 10 are as follows.
a-1 to a-12; The compounds (a-1) to (a-12) obtained in Examples 1 to 12
c-1 to c-5: The compounds (c-1) to (c-5) obtained in Comparative Examples 1 to 5
Photopolymerization initiator: Ethanone, 1-[9-ethyl-6-(2-methylbenzoyl)-9H-carbazol-3-yl]-, 1-(O-acetyloxime) (“IRGACURE OXE-02” manufactured by BASF Japan Ltd.)
Solvent: Cyclopentanone and 4-formylmorpholine

### Evaluation of Properties of Compositions

### (Evaluation of Film Formability of Compositions)

Each of the compositions obtained in Examples 13 to 24 and Comparative Examples 6 to 10 was applied to a silicon substrate using a spin coater, and then dried at 95° C. for 5 minutes to form a 10 μm-thick composition layer on the silicon substrate. Thereafter, the entire surface of the composition layer was exposed at an exposure amount of 500 mJ/cm² at a wavelength of 365 nm and heated at 150° C. for 5 minutes. The surface of the composition layer was visually observed, and the film formability was evaluated according to the following evaluation criteria.
◯ (Excellent): The surface was smooth.
X (Poor): The surface was not smooth, or generated a crack(s) (i.e., a crack(s) in the composition layer).

### (Evaluation of Curability of Composition)

The silicon substrate having each of the composition layers obtained in the above section was immersed in 100 parts of a 2.38% tetramethylammonium hydroxide aqueous solution (Tokusoh SD-1 manufactured by Tokuyama Corporation) for 1 minute, and then the surface of the composition layer was visually observed, and the curability of the composition was evaluated on the basis of solubility according to the following evaluation criteria. The results are shown in Tables 3 and 4.
◯ (Excellent): The surface of the composition layer was not dissolved.
X (Poor): The surface of the composition layer was dissolved.

### (Evaluation of Resolution of Composition)

Each of the compositions obtained in Examples 13 to 24 and Comparative Examples 6 to 10 was applied to a silicon substrate using a spin coater, and then dried at 95° C. for 5 minutes to form a 10 μm-thick composition layer on the silicon substrate. Thereafter, the composition layer was exposed to a light at 500 mJ/cm² using a photomask with a 1:1 width pattern of lines and spaces, which was subsequently immersed in 100 parts of a 2.38% tetramethylammonium hydroxide aqueous solution (Tokusoh SD-1 manufactured by Tokuyama Corporation) for 1 minute. The resolution of the composition was then evaluated according to the following evaluation criteria.
◯ (Excellent): The minimum resolution was less than 15 μm.
Δ (Moderate): The minimum resolution was 15 to 30 μm.
X (Poor): The minimum resolution was 30 μm or more.

### Example 25 (Preparation of Polymer Film and Polybenzoxazole Film of the Invention)

The composition obtained in Example 13 was applied onto a 18 μm-thick copper foil by using an applicator, and then dried at 100° C. for 120 minutes to form a 20 μm-thick composition layer on the copper foil. The composition layer on the copper foil obtained above was exposed to a light using a conveyor UV irradiation device CS30L-1-1 manufactured by GS Yuasa Corporation) so that the exposure amount at a wavelength of 365 nm was 1000 mJ/cm², and then heated at 150° C. for 60 minutes to form a polymer film on the copper foil. The copper foil having the polymer film obtained above was heated at 225° C. for 120 minutes to perform an intramolecular dehydration ring-closing reaction of the polymer. Thereafter, the copper foil was removed by etching to obtain a polybenzoxazole film of the present invention.

### Examples 26 to 36 and Comparative Examples 11 to 15 (Preparation of Polymer Films and Polybenzoxazole Films of the Invention, and Cured Films for Comparison)

Polymer films and polybenzoxazole films of the present invention, and cured films for comparison were obtained in the same manner as in Example 25 except that the composition obtained in Example 13 was changed to each of the compositions obtained in Examples 14 to 24 and Comparative Examples 6 to 10.

### (Evaluation of Dielectric Properties (Dielectric Constant: Dk, Dielectric Dissipation Factor: Df))

Each of the polybenzoxazole films of the present invention obtained in Examples 25 to 36 and the cured films for comparison obtained in Comparative Examples 11 to 15 cut to a length of 60 mm and a width of 3 mm were individually laminated in two or more layers, and then dried in a desiccator filled with silica gel for 12 hours to prepare test pieces each having a film thickness of 50 to 300 μm. Using a vector type network analyzer ADMSO10c1 manufactured by AET, INC. as a measuring instrument and CP531 (10 GHz band resonator) manufactured by Kanto Electronics Application & Development Inc. as a cavity resonator, the dielectric properties of the test pieces obtained above were measured individually by a cavity resonator perturbation method. The measurement conditions were a frequency of 10 GHz and a temperature of 25° C. The results are shown in Tables 3 and 4.

TABLE 3
Components of Example Compositions and Evaluation Results of Cured Films
Composition

| | | [symbol] | Example | | | | | | | | | | | |
|---|---|---|---|---|---|---|---|---|---|---|---|---|---|---|
| Component | Compound | (Average [symbol] number) | 13 | 14 | 15 | 16 | 17 | 18 | 19 | 20 | 21 | 22 | 23 | 24 |
| Compound listed in | a-1 | 5.0 | 100 | | | | | | | | | | | |
| Syn [symbol] Example | a-2 | 3.3 | | 100 | | | | | | | | | | |
| | a-3 | 2.0 | | | 100 | | | | | | | | | |
| | a-4 | 1.3 | | | | 100 | | | | | | | | |
| | a-5 | 1.0 | | | | | 100 | | | | | | | |
| | a-6 | 2.0 | | | | | | 100 | | | | | | |
| | a-7 | 2.0 | | | | | | | 100 | | | | | |
| | a-8 | 2.0 | | | | | | | | 100 | | | | |
| | a-9 | 2.0 | | | | | | | | | 100 | | | |
| | a-10 | 2.0 | | | | | | | | | | 100 | | |
| | a-11 | 2.0 | | | | | | | | | | | 100 | |
| | a-12 | 2.0 | | | | | | | | | | | | 100 |

| | | | | | | | | | | | | | |
|---|---|---|---|---|---|---|---|---|---|---|---|---|---|
| Photopolymerization | [symbol] acure OXE02 | 5 | 5 | 5 | 5 | 5 | 5 | 5 | 5 | 5 | 5 | 5 | 5 |
| [symbol] nitiator | | | | | | | | | | | | | |
| Solvent | Cyclopentanone | 160 | 160 | 160 | 160 | 160 | 160 | 160 | 160 | 160 | 160 | 160 | 160 |
| | 4-formylmorpholine | 30 | 30 | 30 | 30 | 30 | 30 | 30 | 30 | 30 | 30 | 30 | 30 |

Cured film (Polybenzo [symbol] film)

| | | Example | | | | | | | | | | | |
|---|---|---|---|---|---|---|---|---|---|---|---|---|---|
| Properties for Evaluation | | 25 | 26 | 27 | 28 | 29 | 30 | 31 | 32 | 33 | 34 | 35 | 36 |
| Film [symbol] ility | | ◯ | ◯ | ◯ | ◯ | ◯ | ◯ | ◯ | ◯ | ◯ | ◯ | ◯ | ◯ |
| Curability | | ◯ | ◯ | ◯ | ◯ | ◯ | ◯ | ◯ | ◯ | ◯ | ◯ | ◯ | ◯ |
| Resolution | | Δ | ◯ | ◯ | ◯ | ◯ | ◯ | ◯ | ◯ | ◯ | ◯ | ◯ | ◯ |
| Di [symbol] | D [symbol] | 2.41 | 2.4 [symbol] | 2.4 [symbol] | 2.41 | 2. [symbol] 0 | 2.30 | 2.45 | 2. [symbol] | 2. [symbol] | 2. [symbol] 2 | 2.56 | 2.48 |
| properties | D [symbol] | 0.003 [symbol] | 0.003 [symbol] | 0.0037 | 0.0035 | 0.0039 | 0.0037 | 0.0076 | 0.00 [symbol] 2 | 0.0042 | 0.00 [symbol] | 0.00 [symbol] 3 | 0.00 [symbol] 8 |

[symbol]

indicates data missing or illegible when filed

TABLE 4
Components of Comparative Example Compositions and Evaluation Results of Cured Films
Composition

| | | [symbol] | Comparative Example | | | | |
|---|---|---|---|---|---|---|---|
| Component | Compound | (average repetition number) | 6 | 7 | 8 | 9 | 10 |
| Compound listed in | c-1 | 10.0 | 100 | | | | |
| Synthesis Example | c-2 | 0.5 | | 100 | | | |
| | c-3 | 10.0 | | | 100 | | |
| | c-4 | 0.5 | | | | 100 | |
| | c-5 | 1.5 | | | | | 100 |

| | | | | | | |
|---|---|---|---|---|---|---|
| Photopolymerization initiator | [symbol] acure OXE02 | 5 | 5 | 5 | 5 | 5 |
| Solvent | Cyclopentanone | 160 | 160 | 160 | 160 | 160 |
| | 4-formylmorpholine | 30 | 30 | 30 | 30 | 30 |

Cured flim

| | | Comparative Example | | | | |
|---|---|---|---|---|---|---|
| Properties for Evaluation | | 11 | 12 | 13 | 14 | 15 |
| Film formability | | ◯ | X | ◯ | X | ◯ |
| Curability | | X | ◯ | X | ◯ | ◯ |
| Resolution | | X | X | X | X | ( [symbol] ) |
| Dielec [symbol] properties | D [symbol] | 2.31 | ( [symbol] ) | 2.50 | ( [symbol] ) | 2.50 |
| | D [symbol] | 0.0041 | ( [symbol] ) | 0.0067 | ( [symbol] ) | 0.0039 |

The sign ( [symbol] ) represents that any cured film for determination of properties was not obtained.
The sign ( [symbol] ) represents that the cured film was insoluable to the alkaline developer. [symbol]
indicates data missing or illegible when filed

According to the results shown in Table 3, the compositions of Examples 13 to 24 had good film formability, and exhibited excellent curability and resolution. Moreover, the polybenzoxazole films which are the cured films of Examples 25 to 36 obtained using the compositions of Examples 13 to 24 exhibited excellent dielectric properties, despite the fact that they were formed under a heating condition of 225° C. which was a relatively low temperature.

On the other hand, according to the results shown in Table 4, the compositions of Comparative Examples 6 and 8 had insufficient curability, and accordingly, the exposed portion was also dissolved in the alkaline developer, and significantly lowered the resolution. Moreover, the compositions of Comparative Examples 7 and 9 generated cracks after the exposure and had significantly lowered resolution. The composition of Comparative Example 10 exhibited no alkali developability.

The polymer having a structural unit represented by the formula (1) of the present invention is a photosensitive polybenzoxazole precursor having a negative pattern-forming ability, and it allows an alkaline aqueous solution to be used for pattern formation. Therefore, it has become possible to perfectly eliminate industrial waste of organic solvents which have been produced in a very large amount so far. Furthermore, the polybenzoxazole film obtainable finally from the polymer is excellent in film formability and resolution and also excellent in electrical properties (dielectric properties) despite being formed under a heating condition as lower as 225° C. Therefore, it has become possible to use this cured film as a surface protective film, an interlayer insulating film, and an insulating film of a rewiring layer of semiconductors which have been conventionally used. The present invention relates to the resin backbone itself used in the photosensitive resin composition and the method of producing the same. These inventions are based on the totally novel finding, and a person skilled in the art can naturally appreciate that the inventions are unique and highly excellent ones.

Other modifications and variations will be apparent to those skilled in the art in view of the above detailed description of the invention. However, it is evident that such other modifications and variations may be practiced without departing from the spirit and scope of the invention.

1. A compound having unsaturated double bonds represented by the following formula (1): 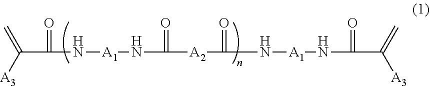
wherein A₁ represents a divalent linking group represented by the following formula (1-1), (1-2), or (1-3): 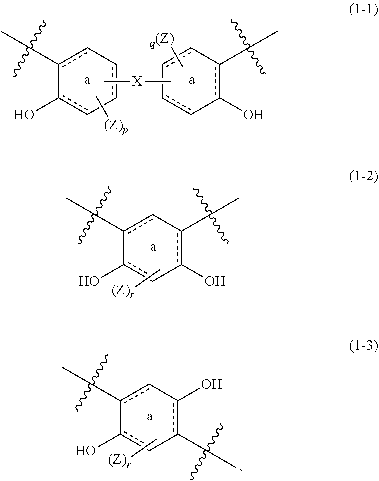
wherein ring a represents a benzene ring or a cyclohexane ring; X represents a direct bond or a divalent linking group, and when two or more Xs are present in the formula (1), the two or more Xs may be the same or different from each other; Z represents a monovalent substituent bonded to ring a, and when two or more Zs are present in the formula (1), the two or more Zs may be the same or different from each other; p, q, and r are numbers of the monovalent substituent Z, p and q each independently represent an integer of 0 to 3, r represents an integer of 0 to 2, and when two or more ps are present in the formula (1-1), the two or more ps may be the same or different from each other, and when two or more qs are present in the formula (1-1), the two or more qs may be the same or different from each other, and when two or more rs are present in the formulae (1-2) and (1-3), the two or more rs may be the same or different from each other,
or a divalent linking group other than the formulae (1-1), (1-2), and (1-3), provided that at least one of A₁ is a divalent linking group represented by the formula (1-1), (1-2), or (1-3); A represents a divalent linking group obtained by removing two carboxy groups from a saturated aliphatic dicarboxylic acid compound, a divalent linking group obtained by removing two carboxy groups from an aromatic dicarboxylic acid compound, or a divalent linking group other than a divalent linking group obtained by removing two carboxy groups from a saturated aliphatic dicarboxylic acid compound or a divalent linking group obtained by removing two carboxy groups from an aromatic dicarboxylic acid compound, and when two or more A₂s are present, the two or more A₂s may be the same or different from each other; A₃ represents a hydrogen atom or a methyl group; and n is an average value of the repeating unit numbers, and is a real number within a range of 1≤n≤9).

2. The compound having unsaturated double bonds according to claim 1, wherein A₁s are each independently a divalent linking group represented by any one of formulae (1-1), (1-2), and (1-3), and each of the ring a is a benzene ring.

3. The compound having unsaturated double bonds according to claim 1, wherein 1.0<n≤4.0.

4. The compound having unsaturated double bonds according to claim 1, wherein at least one of A₂(s) is a divalent linking group obtained by removing two carboxy groups from a saturated aliphatic dicarboxylic acid compound.

5. The compound having unsaturated double bonds according to claim 1, wherein each of A₂(s) is a divalent linking group obtained by removing two carboxy groups from a saturated aliphatic dicarboxylic acid compound.

6. The compound having unsaturated double bonds according to claim 1, wherein each of A₂(s) is a divalent linking group obtained by removing two carboxy groups from a saturated aliphatic dicarboxylic acid compound, and the average number of carbon atoms contained in the aliphatic chain of each A₂ is 2 to 10.

7. The compound having unsaturated double bonds according to claim 1, wherein X is each independently a direct bond or a divalent linking group represented by any one of the following formulae (a) to (f).

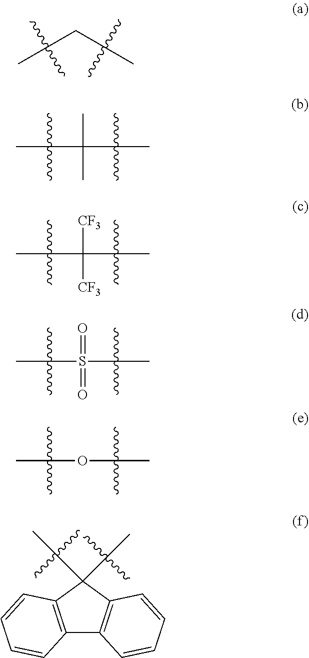

8. A composition containing the compound according to claim 1 and a photopolymerization initiator or a curing catalyst.

9. A composition containing the compound having unsaturated double bonds according to claim 1, and a compound capable of reacting with an unsaturated double bond.

10. A polybenzoxazole which is an intramolecular dehydrated ring-closed product of a polymer of the compound according to claim 1.

11. A semiconductor device provided with a surface protective film, an interlayer insulating film, or an insulating film of a rewiring layer, which contains the polybenzoxazole according to claim 10.

12. A dry film resist, in which a composition containing the compound according to claim 1 is sandwiched between substrates.

13. The compound having unsaturated double bonds according to claim 2, wherein 1.0<n≤4.0.

14. The compound having unsaturated double bonds according to claim 2, wherein at least one of A₂(s) is a divalent linking group obtained by removing two carboxy groups from a saturated aliphatic dicarboxylic acid compound.

15. The compound having unsaturated double bonds according to claim 2, wherein each of A₂(s) is a divalent linking group obtained by removing two carboxy groups from a saturated aliphatic dicarboxylic acid compound.

16. The compound having unsaturated double bonds according to claim 2, wherein each of A₂(s) is a divalent linking group obtained by removing two carboxy groups from a saturated aliphatic dicarboxylic acid compound, and the average number of carbon atoms contained in the aliphatic chain of each A₂ is 2 to 10.

17. The compound having unsaturated double bonds according to claim 2, wherein X is each independently a direct bond or a divalent linking group represented by any one of the following formulae (a) to (f).

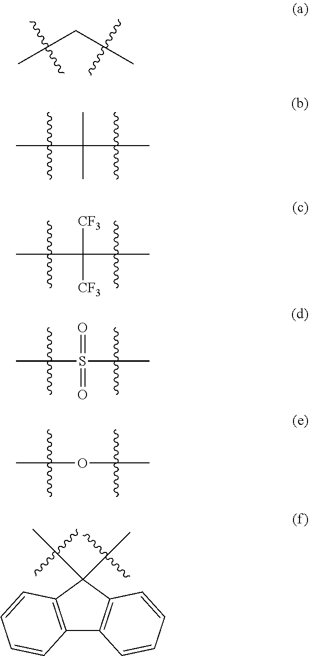

18. A composition containing the compound according to claim 2 and a photopolymerization initiator or a curing catalyst.

19. A composition containing the compound having unsaturated double bonds according to claim 2, and a compound capable of reacting with an unsaturated double bond.

20. A polybenzoxazole which is an intramolecular dehydrated ring-closed product of a polymer of the compound according to claim 2.

21. A semiconductor device provided with a surface protective film, an interlayer insulating film, or an insulating film of a rewiring layer, which contains the polybenzoxazole according to claim 20.

22. A dry film resist, in which a composition containing the compound according to claim 2 is sandwiched between substrates.
